(19) United States
(12) Patent Application Publication
(10) Pub. No.: US 2017/0030068 A1
(43) Pub. Date: Feb. 2, 2017

(54) DECONTAMINATION APPARATUS PROVIDING OZONE AND ITS USE FOR DECONTAMINATING A FLOOR DRAIN

(71) Applicant: ANACAIL LIMITED, Glasgow Lanarkshire (GB)

(72) Inventors: Hugh POTTS, Glasgow Lanarkshire (GB); Declan DIVER, Glasgow Lanarkshire (GB)

(73) Assignee: ANACAIL LIMITED, Glasgow Lanarkshire (GB)

(21) Appl. No.: 15/102,790

(22) PCT Filed: Dec. 19, 2014
(86) PCT No.: PCT/GB2014/053773

§ 371 Date: Jun. 8, 2016

(30) Foreign Application Priority Data
Dec. 19, 2013 (GB) 1322511.5

(51) Int. Cl. E03F 9/00; A61L 2/24; E03F 5/04; A61L 2/20

(52) U.S. Cl. CPC E03F 9/00; A61L 2/202; A61L 2/24; E03F 5/041; A61L 2202/11; A61L 2202/121; A61L 2202/14

(57) ABSTRACT

The present application concerns an apparatus and the corresponding use of a drain decontamination apparatus for decontamination and disinfecting floor drains contaminated with pathogens. The drain decontamination apparatus comprises an open enclosure capable of forming a seal against an area of floor, a dielectric barrier discharge device (2) capable of forming ozone, and sealing means (6) attached to ends of the open enclosure where the sealing means is capable of forming a seal against an area of the floor.

### FIELD OF THE INVENTION

The present invention relates to an apparatus and method for decontaminating and disinfecting. More particularly, the present invention relates to an apparatus and method for decontaminating and disinfecting areas contaminated with pathogens such as drainage systems.

### BACKGROUND OF THE INVENTION

Decontamination of drains and drain pots is a significant problem, particularly in the food industry. Biofilms that accumulate on the surfaces of the drain pipes and grilles can protect pathogens which can recontaminate the room above after cleaning, particularly in a persistently wet environment.

Many biofilms are highly resistant to common chlorine based liquid bleaches—this problem is exacerbated by the difficulty of evenly coating surfaces in an enclosed drain with a liquid disinfectant. Gaseous ozone has been shown to be very effective at removing biofilms, and also due to its gaseous nature is extremely good at giving a uniform treatment of all surfaces regardless of their orientation. Ozone decays rapidly back to oxygen in the presence of organic material, leaving no long-lived chemically active species, reducing any pollution risks associated with putting potent germicidal chemicals down a drain. Ozone is also approximately 1.5 times more dense than air, so any residual ozone at the end of treatment will stay in the drain pot until it spontaneously decomposes.

Therefore, although prior art systems are known for cleaning drainage systems these prior art systems all have associated problems.

It is an object of at least one aspect of the present invention to obviate or mitigate at least one or more of the aforementioned problems.

It is a further object of at least one aspect of the present invention to provide an improved method and apparatus for decontaminating and disinfecting areas contaminated with pathogens such as drainage systems.

### SUMMARY OF THE INVENTION

According to a first aspect of the present invention there is provided a drain decontamination apparatus comprising:

an open enclosure capable of forming a seal against an area of floor;

a dielectric barrier discharge device capable of forming ozone;

sealing means attached to ends of the open enclosure where the sealing means are capable of forming a seal against an area of the floor; and

wherein the drain decontamination apparatus is capable of decontaminating and disinfecting areas contaminated with pathogens.

Generally speaking, the present invention therefore resides in the provision of an apparatus and method for decontaminating and disinfecting areas contaminated with pathogens such as drainage systems. However, the system may be used to treat any other area where localised gaseous decontamination is required.

Alternatively, the drain decontamination apparatus may be used for removing any form of biofilms where there is a drain or pipe with a liquid at the bottom.

The open enclosure may form an open cavity underneath such as in the form of a substantially concave shape (i.e. an inverted bowl). However, any suitable shape that provides a cavity under the apparatus capable of providing decontamination is within the scope of the present invention.

The drain decontamination apparatus may be made from any suitable material such as high impact, ozone resistant plastic.

The drain may be any form of vertical or substantially vertical pipe.

On the upper area of the drain decontamination apparatus there may be located the dielectric barrier discharge device which may optionally be flat or substantially flat. The dielectric barrier discharge device is capable of generating ozone from the atmospheric air.

The dielectric barrier discharge device may be powered by an integral battery-powered high voltage system which controls the ozone dose rate and timing.

The dielectric barrier discharge device and its high voltage power system and batteries may be fully encapsulated, optionally sealed to IPX8 or IP69K, and capable of being power washed with common cleaning chemicals. The surface of barrier discharge device may be impermeable and hydrophobic, and resistant to cleaning chemicals. The dielectric barrier discharge device is made such that it is electrically safe even when operating in wet conditions.

The integral battery-powered high voltage system may be capable of being charged by using an inductively coupled charger system to avoid any external metallic connections.

The drain decontamination apparatus may also have a top mounted handle.

The sealing means may be a resilient multi-lip seal system.

The sealing means may be made from an ozone resistant polymer such as Viton (Registered Trade Mark) or Santoprene (Registered Trade Mark).

The sealing means is capable of forming a tight seal to the floor around the area being treated. The seal may be maintained by gravity due to the weight of the drain decontamination apparatus. Optionally the apparatus may have a system for mechanically attaching it to the drain or floor around the drain to be treated.

The drain decontamination apparatus may also comprise additional weights which may be molded low down into the plastic housing to increase the stability of the drain decontamination apparatus.

In use, the drain decontamination apparatus may be fitted over a drain gate. The drain decontamination apparatus may be started manually and may produce a metered dose of ozone from the electrode system over a set time. The delivery of ozone may follow a pulsed pattern, and may be designed to maintain a high ozone concentration over the full treatment period. The drain decontamination apparatus may be programmable via, for example, Bluetooth (Registered Trade Mark) or some other related wireless mechanism.

The drain decontamination apparatus may also comprise a lighting system such as a red light which is capable of indicating when there is ozone under the drain decontamination apparatus, and a green light will indicate when it is safe to remove.

According to a second aspect of the present invention there is provided a method of decontaminating and disinfecting areas contaminated with pathogens using a drain decontamination apparatus comprising:

an open enclosure capable of forming a seal against an area of floor;

a dielectric barrier discharge device capable of forming ozone;

sealing means attached to ends of the open enclosure where the sealing means is capable of forming a seal against an area of the floor; and

wherein the drain decontamination apparatus is capable of decontaminating and disinfecting areas contaminated with pathogens.

The drain decontamination apparatus may be as defined in the first aspect.

### BRIEF DESCRIPTION OF THE DRAWINGS

Embodiments of the present invention will now be described, by way of example only, with reference to the accompanying drawings in which:

FIG. 1 is a representation of a drain decontamination apparatus according to an embodiment of the present invention;

FIG. 2 is a representation of the drain decontamination apparatus shown in FIG. 1 being carried by a user; and

FIG. 3 is a cross-sectional representation of the drain decontamination apparatus shown in FIGS. 1 and 2 in use.

### BRIEF DESCRIPTION

Generally speaking, the present invention resides in the provision of an apparatus and method for decontaminating and disinfecting areas contaminated with pathogens such as drainage systems.

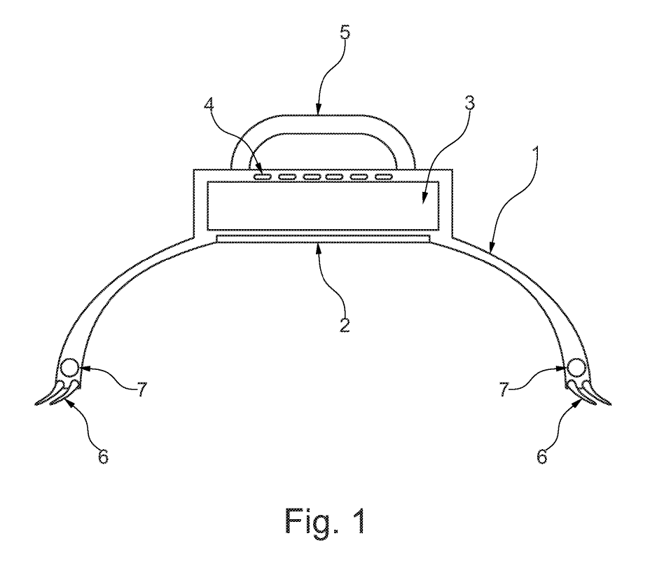

FIG. 1 is a representation of a drain decontamination apparatus according to the present invention generally designated 1. The drain contamination apparatus described in the present application is especially aimed at the problems of disinfecting floor mounted drains in an industrial environment, particularly a wet food processing environment, where regular wash down is essential.

As shown in FIG. 1 the drain decontamination apparatus 1 is of a substantially concave shape (i.e. an inverted bowl). However, any suitable shape that provides a cavity under the apparatus 1 capable of providing decontamination is also with the scope of the present invention. The drain decontamination apparatus 1 is made from any suitable material such as high impact, ozone resistant plastic. The surface of the drain decontamination apparatus 1 is impermeable and hydrophobic.

Onto the upper area of the drain decontamination apparatus 1 there is located a dielectric barrier discharge device 2 which may as shown in FIG. 1 be flat or substantially flat. The dielectric barrier discharge device 2 is capable of generating ozone from the atmospheric air.

The dielectric barrier discharge device 2 is powered by an integral battery-powered high voltage system 3 which controls the ozone dose rate and timing. The dielectric barrier discharge device 2 is fully encapsulated, optionally sealed to IPX8 or IP69K, and capable of being power washed with common cleaning chemicals. The integral battery-powered high voltage system 3 is capable of being charged by using an inductively coupled charger system 4 to avoid any external metallic connections.

As shown in FIG. 1, the drain decontamination apparatus 1 also comprises a top mounted handle 5 and a resilient multi-lip seal system 6. The resilient multi-lip seal system 6 is made from an ozone resistant polymer such as Viton (Registered Trade Mark) or Santoprene (Registered Trade Mark). The resilient multi-lip seal system 6 is capable of forming a tight seal to the floor around the area being treated. The seal is maintained by gravity due to the weight of the drain decontamination apparatus 1. Optionally additional weights 7 may be molded low down into the plastic housing to increase the stability of the drain decontamination apparatus 1, and reduce the risk of it being accidentally knocked off the drain.

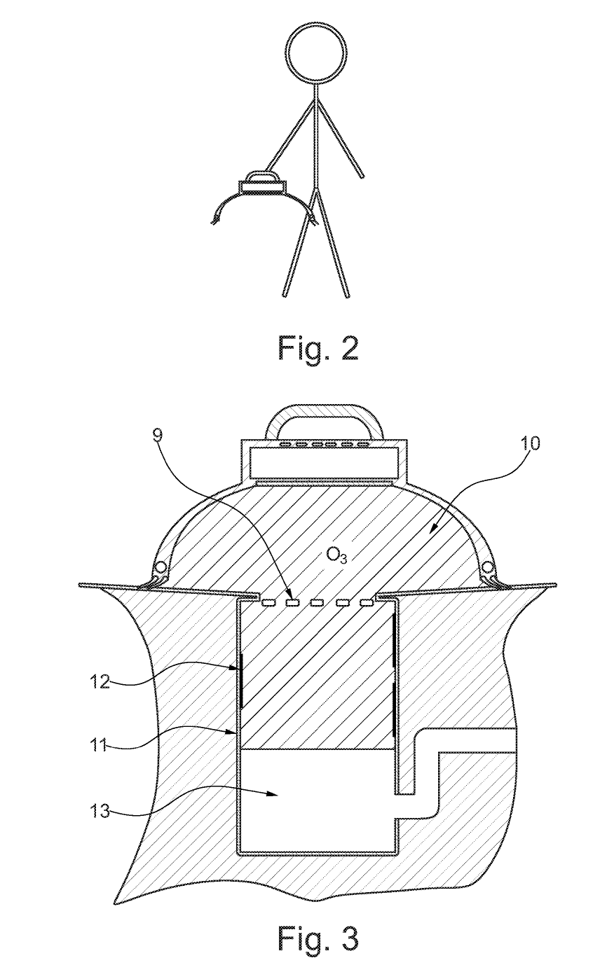

FIG. 2 shows the drain decontamination apparatus 1 being carried by a user using the handle 5.

FIG. 3 shows the drain decontamination apparatus 1 in use. The drain decontamination apparatus 1 is fitted over a drain gate 9. The drain decontamination apparatus 1 can be started manually and will produce a metered dose of ozone from the electrode system over a set time. The delivery of ozone will follow a pulsed pattern, and be designed to maintain a high ozone concentration over the full treatment period. The ozone dose will be tailored to the volume of the drain system being treated. The drain decontamination apparatus 1 may be programmable via, for example, Bluetooth (Registered Trade Mark) or some other related wireless mechanism.

As ozone is denser than air, the ozone 10 will sink into the drain system 11 and diffuse into the biofilms 12, damaging their structural integrity, and destroying any pathogens within. Ozone will also dissolve into the fluid within the drain trap 13.

The drain decontamination apparatus 1 may comprise a lighting system such as a red light which is capable of indicating when there is ozone under the drain decontamination apparatus 1, and a green light will indicate when it is safe to remove. Removal while active would vent a small but non-hazardous amount of ozone into the atmosphere, and the generator would automatically stop. The drain decontamination apparatus 1 would be used regularly as part of the normal cleaning procedures.

Whilst specific embodiments of the present invention have been described above, it will be appreciated that departures from the described embodiments may still fall within the scope of the present invention. For example, any suitable size of drain decontamination apparatus may be envisaged which may be used to provide decontamination. The drain decontamination apparatus may be of a smaller domestic scale to be used in the household.

1-25. (canceled)

26. A drain decontamination apparatus comprising:
an open enclosure capable of forming a seal against an area of floor;
a dielectric barrier discharge device capable of forming ozone;
sealing means attached to ends of the open enclosure where the sealing means is capable of forming a seal against an area of the floor;
wherein the drain decontamination apparatus is capable of decontaminating and disinfecting areas contaminated with pathogens.

27. A drain decontamination apparatus according to claim 26, wherein the drain decontamination apparatus is capable of decontaminating and disinfecting areas contaminated with pathogens in drainage systems and wet food processing environments where regular wash down is essential.

28. A drain decontamination apparatus according to claim 26, wherein the open enclosure forms an open cavity underneath when placed on the ground.

29. A drain decontamination apparatus according to claim 26, wherein the open enclosure forms an open cavity underneath in the form of a substantially concave shape when placed on the ground.

30. A drain decontamination apparatus according to claim 26, wherein the drain decontamination apparatus is made from any suitable high impact, ozone resistant plastic.

31. A drain decontamination apparatus according to claim 26, wherein the surface of the dielectric barrier discharge device is impermeable and hydrophobic and resistant to all common cleaning chemicals.

32. A drain decontamination apparatus according to claim 26, wherein on the upper area of the drain decontamination apparatus there is located the dielectric barrier discharge device.

33. A drain decontamination apparatus according to claim 26, wherein the dielectric barrier discharge device is capable of generating ozone from the atmospheric air.

34. A drain decontamination apparatus according to claim 26, wherein the dielectric barrier discharge device is powered by an integral battery-powered high voltage system which controls the ozone dose rate and timing.

35. A drain decontamination apparatus according to claim 26, wherein the entire system is fully encapsulated, and capable of being power washed with common cleaning chemicals.

36. A drain decontamination apparatus according to claim 26, wherein the integral battery-powered high voltage system is capable of being charged by using an inductively coupled charge system to avoid any external metallic connections.

37. A drain decontamination apparatus according to claim 26, wherein the drain decontamination apparatus comprises a top mounted handle.

38. A drain decontamination apparatus according to claim 26, wherein the sealing means is a resilient multi-lip seal system.

39. A drain decontamination apparatus according to claim 26, wherein the sealing means is made from an ozone resistant polymer.

40. A drain decontamination apparatus according to claim 26, wherein the sealing means is capable of forming a seal to the floor around the area being treated.

41. A drain decontamination apparatus according to claim 26, wherein the drain decontamination apparatus also comprises additional weights which are capable of being molded low down into the plastic housing to increase the stability of the drain decontamination apparatus.

42. A drain decontamination apparatus according to claim 26, wherein the decontamination apparatus is clamped or mechanically attached to the floor using fittings mounted to the drain or floor; and wherein there is a drain cover specifically designed for the decontamination apparatus to be attached to.

43. A method of decontaminating and disinfecting areas contaminated with pathogens using a drain decontamination apparatus comprising:
an open enclosure capable of forming a seal against an area of floor;
a dielectric barrier discharge device capable of forming ozone;
sealing means attached to ends of the open enclosure where the sealing means is capable of forming a seal against an area of the floor; and
wherein the drain decontamination apparatus is capable of decontaminating and disinfecting areas contaminated with pathogens.

44. A method of decontaminating and disinfecting areas contaminated with pathogens using a drain decontamination apparatus according to claim 43, wherein in use, the drain decontamination apparatus is fitted over a drain grate; wherein the drain decontamination apparatus is capable of being started manually and produces a metered dose of ozone from the electrode system over a set time; and wherein the delivery of ozone follows a pulsed pattern, and is designed to maintain a high ozone concentration over the full treatment period.

45. A method of decontaminating and disinfecting areas contaminated with pathogens using a drain decontamination apparatus according to claim 43, wherein the drain decontamination apparatus is programmable via, for example, Bluetooth (Registered Trade Mark) or some other related wireless mechanism; and wherein the drain decontamination apparatus comprises a lighting system which is capable of indicating when there is ozone under the drain decontamination apparatus, and when the apparatus is safe to be removed.
